(19) United States
(12) Patent Application Publication
(10) Pub. No.: US 2025/0060308 A1
(43) Pub. Date: Feb. 20, 2025

(54) DEVICE AND METHOD FOR THE OPTICAL ASSESSMENT OF THE LEVEL OF HEMOLYSIS IN A BLOOD SAMPLE

(71) Applicants: Giorgio PITRUZZELLO, Massa (IT); SCUOLA SUPERIORE DI STUDI UNIVERSITARI E DI PERFEZIONAMENTO SANT'ANNA, Pisa (IT); ABZERO SRL, Pisa (IT)

(72) Inventors: Giorgio PITRUZZELLO, Massa (IT); Giuseppe Roberto TORTORA, Pisa (IT)

(73) Assignees: Giorgio PITRUZZELLO, Massa (IT); SCUOLA SUPERIORE DI STUDI UNIVERSITARI E DI PERFEZIONAMENTO SANT'ANNA, Pisa (IT); ABZERO SRL, Pisa (IT)

(21) Appl. No.: 18/723,284

(22) PCT Filed: Dec. 23, 2022
(86) PCT No.: PCT/IB2022/062729

§ 371 Date: Jun. 21, 2024

(30) Foreign Application Priority Data
Dec. 23, 2021 (IT) 102021000032501

(51) Int. Cl. G01N 21/31; G01N 33/49

(52) U.S. Cl. CPC G01N 21/31; G01N 33/49

(57) ABSTRACT

A method for the optical evaluation of the level of hemolysis in a blood sample, said method comprising the steps of prearranging a device 100 comprising a light emitter, at least two photodetectors and a control unit arranged to operate the light emitter and to receive data by the photodetectors. The method then comprises a step of emitting, by the light emitter, of an inspection electromagnetic radiation towards the blood sample, said inspection electromagnetic radiation comprising a plurality of inspection electromagnetic waves having respective wavelengths λ_{E}. A step follows of receiving, by the photodetectors, a return electromagnetic radiation coming from the blood sample, said return electromagnetic radiation comprising a plurality of return electromagnetic waves having respective wavelengths λ_{R}.

### FIELD OF THE INVENTION

The present invention belongs to the technical sector concerning devices for evaluating the level of hemolysis in the blood.

### DESCRIPTION OF THE PRIOR ART

Blood represents a public good and, given the high perishability and scarcity of plasma and blood products due to the few annual donations, it should be considered as a good of primary importance.

In the healthcare sector, one of the main challenges is that relating to the logistical and management aspects of the transport of blood and blood products, in order to ensure that this transport is rapid, efficient and operationally compliant with the stringent regulations in force.

To date, the operating conditions in which the service is provided mean that over 40% of transports do not comply with the conditions required by the legislation, constantly putting the quality of the transported goods at risk, with the related negative impacts on the health of the patients and costs for the health system. In some cases, especially when the blood is subjected to stress (high temperatures, mechanical agitation), red blood cells can undergo uncontrolled hemolysis, a process that leads to the destruction of the cell. When red blood cells break down, they leak into the serological component of the blood, the plasma, or into the storage fluid if the hemolysis occurs in a blood bag.

Each bag or sample of blood transported beyond the temperature limits is therefore potentially at risk of haemolysis and requires an additional test at the destination centre.

Various methodologies for evaluating the level of hemolysis in the blood are known, including methodologies based on optical and therefore non-invasive principles.

Document JPS64450A describes a system for evaluating the level of hemolysis comprising a light emitting diode and a photosensor suitable for being arranged on the opposite side with respect to a small tube in which plasma flows. The diode is configured to emit a green light, while the photosensor detects the light transmitted through the plasma and emits an electrical output signal, which is then amplified and analysed by a voltage comparator to verify the level of hemolysis.

However, this system is based on the emission of a single wavelength, which allows a reduced ability to analyse the haemolytic state of the blood and is easily subject to background noise in the generated electrical signal.

### SUMMARY OF THE INVENTION

It is therefore a feature of the present invention to provide a method for the optical evaluation of the level of hemolysis in a blood sample which allows to recognize a plurality of wavelengths of interest and generate corresponding electrical signals.

It is also a feature of the present invention to provide such a method which allows the intensity of the radiation to be calibrated as function of the generated electrical signal, reducing the background noise of the electrical signal itself.

It is still a feature of the present invention to provide such a method which allows to evaluate the level of hemolysis both by means of the absorbance phenomenon and by means of the reflection phenomenon.

It is a further feature of the present invention to provide such a method which also provides for a preliminary step of separation of the liquid part from the corpuscular part of the blood, facilitating the optical evaluation of the level of hemolysis.

It is therefore a feature of the present invention to provide a device that carries out this method.

These and other objects are achieved by a method for the optical evaluation of the level of hemolysis in a blood sample, said method comprising the steps of:
prearranging a device for the optical evaluation of the level of hemolysis comprising:
   a light emitter;
   at least two photodetectors;
   a control unit arranged to operate the light emitter and to receive data by the photodetectors;
prearranging a blood sample to be analysed;
emitting, by the light emitter, an inspection electromagnetic radiation towards the blood sample, said inspection electromagnetic radiation comprising a plurality of inspection electromagnetic waves having respective wavelengths λ_{E};
receiving, by the photodetectors, a return electromagnetic radiation coming from the blood sample, said return electromagnetic radiation comprising a plurality of return electromagnetic waves having respective wavelengths λ_{R};
generating, by the photodetectors, two respective electric signals associated with return electromagnetic waves having wavelengths of interest λ_{R1}* and λ_{R2}*, said electric signals generated having an intensity of current or a voltage proportional to the return electromagnetic waves having the wavelengths of interest λ_{R1}* and λ_{R2}*;
calculating, by the control unit, a value of the level of hemolysis in the blood sample on the basis of the electric signals generated.

Thanks to the use of two photodetectors, it is possible to carry out two distinct measurements which, combined together, allow to obtain a final data which is more robust with respect to optical disturbances which may occur, such as for example scattering. In this way, considerably greater precision is obtained than in the prior art in the final calculation of the hemolysis value in the blood sample.

Advantageously, is also provided, by the control unit, a step of modulating the intensity of the inspection electromagnetic radiation as function of the level of hemolysis calculated.

In particular, the modulating step ends when the intensity of the electromagnetic signals has a predetermined value associated with the value of the level of hemolysis calculated.

In this way, through an iterative process, the intensity of the electromagnetic radiation emitted is calibrated according to the electrical signal produced by the photodetector, reducing the background noise of the electrical signal itself.

In particular, the wavelengths of interest λ_{R1}* and λ_{R2}* are comprised between 400 and 600 nm.

Advantageously, the device for the optical evaluation comprises a plurality of photodetectors adapted to generate respective signals associated with return electromagnetic waves having wavelengths of interest.

Advantageously, the wavelength of interest λ_{R1}* is comprised between 520 and 550 nm.

In particular, λ_{R1}* is equal to 540 nm. Such wavelength corresponds to an absorption peak of haemoglobin in the visible spectrum.

Advantageously, the wavelength of interest λ_{R2}* is comprised between 550 and 590 nm.

In particular, λ_{R2}* is equal to 575 nm. Such wavelength corresponds to another peak of haemoglobin absorption in the visible spectrum.

In particular, a third photodetector is provided adapted to generate a third electric signal associated with a return electromagnetic wave having a wavelength of interest λ_{R3}* equal to about 415 nm. This value corresponds to the highest peak of haemoglobin absorption.

In particular, λ_{R1}* is comprised between 520 and 550 nm, λ_{R2}* is comprised between 550 and 590 nm and λ_{R3}* is about 415 nm.

Advantageously, in the calculation step, the value of the level of hemolysis is a value inversely proportional to the intensity of current or to the voltage of the electric signal generated. This way, the level of hemolysis is calculated by the phenomenon of the absorbance, i.e. the level of hemolysis is calculated as function of the amount of light adsorbed by the blood sample during the emission step.

Advantageously, in the calculation step, the value of the level of hemolysis is a value calculated on the basis of the values of the intensity of current or the voltage of the electric signals generated.

In particular, the level of hemolysis is calculated by the values of intensity of current weighted with respect to predetermined coefficients.

More in particular, the level of hemolysis is calculated by the equation:

$${\sum }_{i = 1}^{n} {C}_{i} * R ( {λ}_{R i}^{*} )$$

where Cᵢ is the i-th experimental coefficient, set between 0 and 1, and R(λ_{Ri}*) is the voltage (converted into mg/dl) of the electric signal associated with the i-th wavelength of interest λ_{Ri}*.

In particular, the level of hemolysis is calculated by the experimental formula:

$$154.7 * R ( {λ}_{R 1}^{*} ) + 130.7 * R ( {λ}_{R 2}^{*} ) + 154.7 * R ( {λ}_{R 3}^{*} )$$

where λ_{R1}*=415 nm, λ_{R2}*=450 nm, λ_{R3}*=700 nm

In particular, in the calculation step, the value of the level of hemolysis is a value directly proportional to the intensity of current or to the voltage of the electric signal generated. This way, the level of hemolysis is calculated by the phenomenon of the reflection, i.e. the level of hemolysis is calculated as function of the amount of light reflected by the blood sample during the emission step.

Advantageously, the device for the optical evaluation comprises a wireless antenna and a step is provided of emitting, by means of this wireless antenna, data concerning the evaluation of the value of the level of hemolysis in the blood sample.

Advantageously, a step is provided of separating the liquid component by the particle component in the blood sample.

In particular, the separation step is made by gravity.

In particular, the separation step is performed by externally compressing a bag containing the blood sample and a filtering membrane, so as to make the liquid component flow beyond the filtering membrane to separate it from the corpuscular component.

Alternatively, the separation step is carried out by automated methodologies, for example by stirring, by means of a centrifuge or by means of a dedicated pump.

Advantageously, the control unit also suitable for calculating additional parameters, such as for example the temperature, the pH, the humidity or the spatial acceleration of the blood bag.

In particular, according to an aspect of the invention, the devices placed on relative bags in the same environment can wirelessly communicate the monitored parameters to an artificial intelligence, which processes them so as to calculate the overall quality of the blood sample of each bag, in order to help the medical operator in choosing the bag to use.

Such wireless communication may take place, for example, via Bluetooth Low Energy and/or Wi-Fi.

Advantageously, thanks to the wireless communication of the parameters monitored by the various devices, artificial intelligence can also provide the user with information on the punctual distribution of a specific parameter (e.g. temperature) in the considered environment.

According to another aspect of the invention, a device for the optical evaluation of the level of hemolysis is claimed comprising:
a light emitter arranged to emit an inspection electromagnetic radiation towards the blood sample, said inspection electromagnetic radiation comprising a plurality of inspection electromagnetic waves having respective wavelength λ_{E};
at least two photodetectors arranged to:
   receive a return electromagnetic radiation coming from the blood sample, said return electromagnetic radiation comprising a plurality of return electromagnetic waves having respective wavelengths λ_{R};
   generate two electric signals associated with return electromagnetic waves having wavelengths of interest λ_{R1}* and λ_{R2}*, said electric signals generated having an intensity of current or a voltage proportional to the return electromagnetic waves having the wavelengths of interest λ_{R1}* and λ_{R2}*;
a control unit arranged to:
   operate the light emitter and receive data from the photodetectors;
   calculate a value of the level of hemolysis in the blood sample on the basis of the electric signals generated.

### BRIEF DESCRIPTION OF THE DRAWINGS

The invention will be now shown with the following description of some exemplary embodiments, exemplifying but not limitative, with reference to the attached drawings in which:

FIG. 1 shows a possible embodiment of the device for the optical evaluation of the level of hemolysis in a blood sample, according to the present invention, during the reading of a blood sample inside a bag;

FIG. 2 schematically shows the following steps of the method for the optical evaluation of the level of hemolysis in a blood sample, according to the present invention;

FIG. 3 shows a possible embodiment of the device applied in three different points of a blood bag.

### DESCRIPTION OF SOME PREFERRED EXEMPLARY EMBODIMENTS

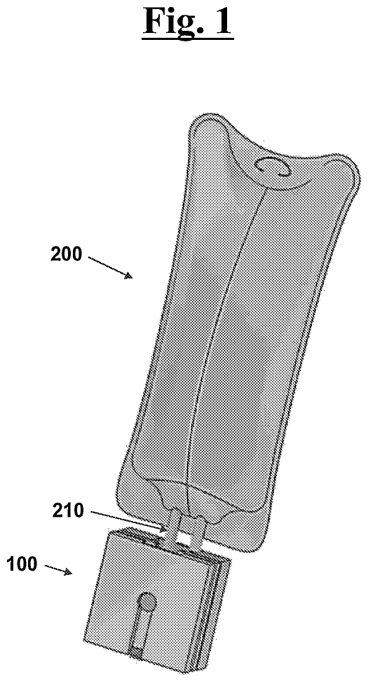

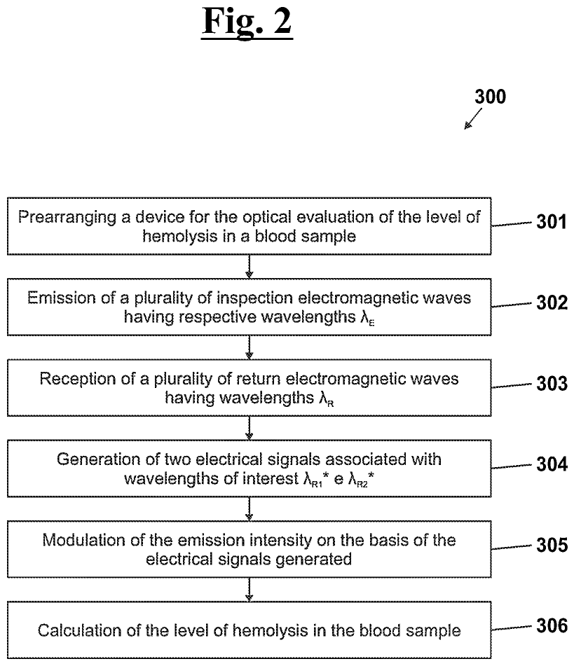

With reference to FIGS. 1 and 2, the method for the optical evaluation of the level of hemolysis in a blood sample, according to the present invention, comprises a first step of prearranging a device 100 arranged to evaluate the level of hemolysis of a blood sample, for example located in a bag 200 [301].

In particular, the device 100 comprises a light emitter, two photodetectors and a control unit.

In FIG. 1 a possible embodiment is shown of the device 100 during the reading of a blood sample in a bag 200. In particular, in this figure, the device 100 is arranged near the tube 210, where the blood was drawn by gravity, by a preliminary step of the method. Advantageously, between the main body of the bag 200 and the tube 210, a filtering membrane can be placed which is able to let only the liquid component of the blood flow into the tube 210, separating it from the corpuscular component, so as to allow a more accurate assessment of the level of hemolysis.

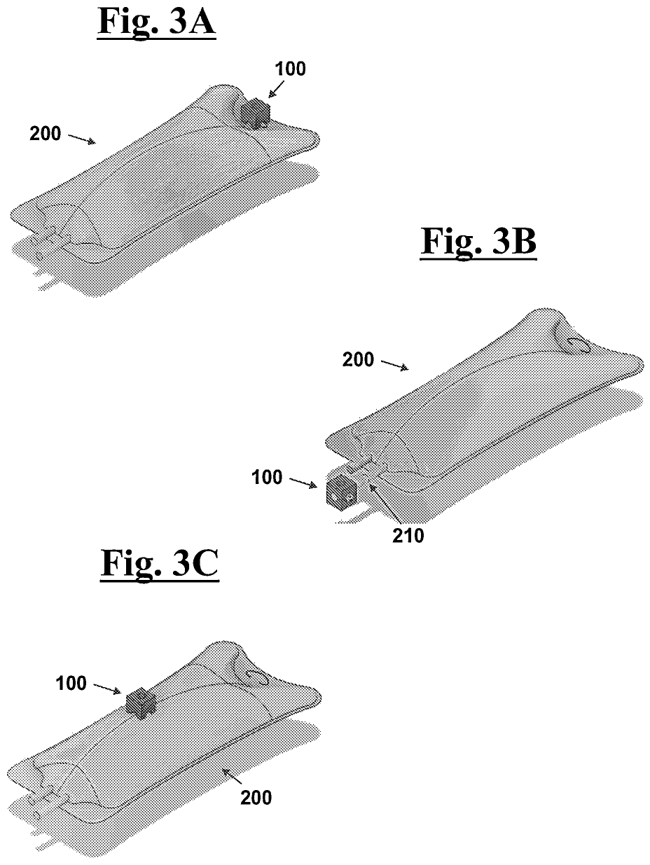

FIGS. 3A, 3B and 3C show another possible embodiment of the device 100 positioned close to a blood bag 200 in three different possible application points. In particular, in FIG. 3B the device 100 is arranged in the lower part of the bag 200, near the tube 210, like in FIG. 1, whereas in FIGS. 3A and 3C the device 100 is arranged, respectively, in the upper part and in the central part of the bag 200.

Advantageously, the control unit is suitable for activating the device 100 when the device is positioned in the vicinity of a blood bag 200. This activation can take place by means of an activation sensor, such as for example an accelerometer, a hall effect sensor, a weight sensor, or other type of sensor. In particular, this activation sensor determines the activation of the device 100 when the latter is moved and subsequently hooked to the bag 200.

This activation sensor can also be integrated with an artificial intelligence or a microprocessor which manages, by means of wireless signals or recognitions (images, QR code, RFID), the procedure for hooking the device 100 to the bag 200, simplifying the quality control of the medical material to be monitored.

Once the device 100 is placed in proximity to the blood sample, the method provides for a step of emission, by means of the light emitter, of an electromagnetic radiation comprising a plurality of inspection electromagnetic waves having respective wavelengths λ_{E} [302].

Thus follows a step of receiving a return electromagnetic radiation comprising a plurality of return electromagnetic waves having respective wavelengths λ_{R} [303].

There is therefore a step of generation, by means of the photodetectors, of two electrical signals associated with return electromagnetic waves having wavelengths of interest λ_{R1}* and λ_{R2}* [304].

In particular, the electrical signals generated have a current intensity or a voltage proportional to the return electromagnetic waves having said wavelengths of interest λ_{R1}* and λ_{R2}*.

The method then comprises a step of calculation, by means of the control unit, of a value of the hemolysis level in the blood sample on the basis of the generated electrical signals [306].

Furthermore, according to an embodiment of the invention, the method also comprises a step of modulating the intensity of the inspection electromagnetic radiation as a function of the calculated hemolysis level.

In particular, this modulation step is obtained through an iterative process which ends when the intensity of the electromagnetic radiation has a predetermined value associated with the value of the calculated hemolysis level.

In a further embodiment of the invention, not shown in the figures, the device 100 can be completely integrated into the bag 200 making this bag 200 become a device of the “Internet of Things” (IoT) type.

The foregoing description exemplary embodiments of the invention will so fully reveal the invention according to the conceptual point of view, so that others, by applying current knowledge, will be able to modify and/or adapt for various applications such embodiment without further research and without parting from the invention, and, accordingly, it is therefore to be understood that such adaptations and modifications will have to be considered as equivalent to the specific embodiments. The means and the materials to realise the different functions described herein could have a different nature without, for this reason, departing from the field of the invention. It is to be understood that the phraseology or terminology that is employed herein is for the purpose of description and not of limitation.

1. A method for the optical evaluation of the level of hemolysis in a blood sample, said method comprising the steps of:
prearranging a device (100) for the optical evaluation of the level of hemolysis comprising:
a light emitter;
at least two photodetectors;
a control unit arranged to operate said light emitter and to receive data from said photodetectors;
prearranging a blood sample to be analysed;
emitting, by said light emitter, an inspection electromagnetic radiation towards said blood sample, said inspection electromagnetic radiation comprising a plurality of inspection electromagnetic waves having respective wavelengths λ_{E};
receiving, by said photodetectors, a return electromagnetic radiation coming from said blood sample, said return electromagnetic radiation comprising a plurality of return electromagnetic waves having respective wavelengths λ_{R};
generating, by said photodetectors, two respective electric signals associated with return electromagnetic waves having wavelengths of interest λ_{R1}* and λ_{R2}*, said electric signals generated having an intensity of current or a voltage proportional to said return electromagnetic waves having said wavelengths of interest λ_{R1}* and λ_{R2}*;
calculating, by said control unit, a value of the level of hemolysis in said blood sample on the basis of said electric signals generated.

2. The method for the optical evaluation of the level of hemolysis in a blood sample, according to claim 1, wherein a step is also provided, by said control unit, of modulating the intensity of said inspection electromagnetic radiation as function of said level of hemolysis calculated, said modulating step ending when the intensity of said inspection electromagnetic radiation has a predetermined value associated with the value of the level of hemolysis calculated.

3. The method for the optical evaluation of the level of hemolysis in a blood sample, according to claim 1, wherein said wavelengths of interest λ_{R1}* and λ_{R2}* are comprised between 400 and 600 nm.

4. The method for the optical evaluation of the level of hemolysis in a blood sample, according to claim 1, wherein said wavelength of interest λ_{R1}* is comprised between 520 and 550 nm and wherein said wavelength of interest λ_{R2}* is comprised between 550 and 590 nm.

5. The method for the optical evaluation of the level of hemolysis in a blood sample, according to claim 4, wherein, in said step of calculating, said value of the level of hemolysis is a value inversely proportional to said intensity of current or to said voltage of said electric signals generated.

6. The method for the optical evaluation of the level of hemolysis in a blood sample, according to claim 1, wherein said wavelength of interest λ_{R1}* is about 415 nm and wherein said wavelength of interest λ_{R2}* is comprised between 520 and 550 nm.

7. The method for the optical evaluation of the level of hemolysis in a blood sample, according to claim 6, wherein, in said step of calculating, said value of the level of hemolysis is a value directly proportional to said intensity of current or to said voltage of said electric signals generated.

8. The method for the optical evaluation of the level of hemolysis in a blood sample, according to claim 1, wherein said device for the optical evaluation comprises a wireless antenna and wherein a step is provided of emitting, by said wireless antenna, data concerning said evaluation of the value of the level of hemolysis in said blood sample.

9. A device (100) for the optical evaluation of the level of hemolysis comprising:
a light emitter arranged to emit an inspection electromagnetic radiation towards said blood sample, said inspection electromagnetic radiation comprising a plurality of inspection electromagnetic waves having respective wavelengths λ_{E};
at least two photodetectors arranged to:
receive a return electromagnetic radiation coming from said blood sample, said return electromagnetic radiation comprising a plurality of return electromagnetic waves having respective wavelengths λ_{R};
generate two electric signals associated with return electromagnetic waves having wavelengths of interest λ_{R1}* and λ_{R2}*, said electric signals generated having an intensity of current or a voltage proportional to said return electromagnetic waves having said wavelengths of interest λ_{R1}* and λ_{R2}*;
a control unit arranged to:
operate said light emitter and receive data from said photodetectors;
calculate a value of the level of hemolysis in said blood sample on the basis of said electric signals generated.
